(19) United States
(12) Patent Application Publication
(10) Pub. No.: US 2025/0197324 A1
(43) Pub. Date: Jun. 19, 2025

(54) AROMATIC TRANSALKYLATION PROCESS

(71) Applicant: UOP LLC, Des Plaines, IL (US)

(72) Inventors: Kiran Ladkat, Haryana (IN); Jason T. Corradi, Elgin, IL (US); Gregory R. Werba, Arlington Heights, IL (US); Sourabh Maithil, Haryana (IN)

(21) Appl. No.: 18/914,558

(22) Filed: Oct. 14, 2024

Related U.S. Application Data
(60) Provisional application No. 63/611,580, filed on Dec. 18, 2023.

(51) Int. Cl. C07C 7/09; C07C 7/00; C07C 7/10; C07C 7/144

(52) U.S. Cl. CPC C07C 7/09; C07C 7/005; C07C 7/10; C07C 7/144

(57) ABSTRACT

Aromatic transalkylation processes are described which reduce the hydrogen makeup gas requirement to the aromatic transalkylation unit and improve the energy efficiency of the aromatics complex. The processes may utilize a hot separator, optionally one or more flash drums, and optionally a stripper column. The aromatic transalkylation separator bottom liquid may be preheated and flashed to a low-pressure drum to separate light hydrocarbons, such C₁ to C₅, as a vapor stream from the aromatics rich liquid hydrocarbons stream. A portion of flash drum liquid may recycled back to a product condenser inlet as a sponge liquid to absorb the light hydrocarbons from the reactor effluent stream and thereby improve the hydrogen purity of the recycle gas (RG) without purging RG.

### RELATED APPLICATIONS

This application claims priority to U.S. Provisional Patent Application Ser. No. 63/611,580, filed on Dec. 18, 2023, the entirety of which is incorporated herein by reference.

### BACKGROUND

A typical aromatics complex includes an aromatic transalkylation reactor where toluene and C₉-C₁₁ aromatics preferably C₉-C₁₀ aromatics are transalkylated in the presence of hydrogen to form xylenes. The reactor effluent comprises benzene, toluene, xylenes, C₁-C₅ light hydrocarbons, and hydrogen. The reactor effluent is separated in a product separator into an overhead gas stream comprising the C₁-C₅ light hydrocarbons and the hydrogen, and a bottom liquid stream which comprises the benzene, toluene, unconverted aromatic compounds having 9-11 carbon atoms preferably C₉-C₁₀ aromatics and xylenes. The product separator bottom liquid stream is separated in a stripper column into an overhead stream comprising part of the benzene, non-aromatic compounds, and the light hydrocarbons, and a bottoms stream comprising the remaining part of benzene, toluene, unconverted C₉-C₁₁ aromatics and xylenes. The stripper overhead gas stream is used as fuel gas. The stripper bottoms stream is sent to a benzene/toluene (BT) column for further separation into a benzene stream, a toluene stream, and unconverted C₉-C₁₁ aromatics and xylenes stream.

The product separator overhead stream is recycled to the aromatic transalkylation reactor. The recycle hydrogen stream requires a minimum recycle gas hydrogen purity of 55-95 mole % preferably 75 mol % in the reactor section. In order to maintain this hydrogen purity level, a significant purge of the recycle stream is needed. The purge gas stream, which contains a significant amount of hydrogen, is used as fuel gas, and the hydrogen is lost.

In addition, the stripper reboiler consumes a significant amount of high-end energy to separate the C₆ range non-aromatics and part of the benzene from the remaining part of benzene, toluene and xylene products.

Therefore, there is a need for an aromatic transalkylation process which reduces the operating cost of the aromatics complex.

### BRIEF DESCRIPTION OF THE DRAWINGS

FIG. 1 is one embodiment of the aromatic transalkylation process of the present invention.

FIG. 2 is another embodiment of the aromatic transalkylation process of the present invention.

FIG. 3 is another embodiment of the aromatic transalkylation process of the present invention.

FIG. 4 is another embodiment of the aromatic transalkylation process of the present invention.

FIG. 5 is another embodiment of the aromatic transalkylation process of the present invention.

### DESCRIPTION

The present invention meets this need by providing aromatic transalkylation processes which reduce the hydrogen makeup gas requirement to the aromatic transalkylation unit and improve the energy efficiency of the aromatics complex. The processes eliminate the recycle gas purge while maintaining the minimum hydrogen purity of 55-95 mole %, preferably 75%, thereby reducing the hydrogen makeup cost.

In some embodiments, the process utilizes a hot separator, optionally one or more flash drums, and optionally a stripper column. In some processes, the aromatic transalkylation separator bottom liquid is preheated and flashed to a low-pressure drum to separate light hydrocarbons, such C₁ to C₅, as a vapor stream from the aromatics rich liquid hydrocarbons stream. A portion of flash drum liquid is recycled back to a product condenser inlet as a sponge liquid to absorb the light hydrocarbons from the reactor effluent stream and thereby improve the hydrogen purity of the recycle gas (RG) without purging RG. With this arrangement, it is possible to achieve recycle gas purity of 75 mole % with zero purge.

In some embodiments, the processes include a hot separator in the transalkylation reactor section which eliminates the aromatic transalkylation stripper column and associated equipment. The hot flash and cold flash drum liquid is heated in a series of heat exchangers and fed directly to the downstream BT column. The process takes advantage of the bulk separation in the BT column reboiler duty due to inclusion of the aromatic transalkylation hot separator and flash drum. This reduces the fuel consumption in the fired heaters.

In some embodiments, the aromatic transalkylation stripper and associated equipment are replaced with a BT side-cut stripper column to achieve the benzene product specifications (e.g., mass purity of 99.94 wt %). The separation between C₆ aromatics and non-aromatics is done in the low operating pressure BT column and side-cut stripper instead a high operating pressure aromatic transalkylation stripper. The side-cut column also receives extract from the sulfolane extraction unit.

In some embodiments, the process comprises: transalkylating a combined feed stream comprising toluene, C₉-C₁₁ aromatics (preferably C₉-C₁₀ aromatics), and hydrogen forming a reactor effluent stream comprising benzene, xylenes, toluene, unconverted C₉-C₁₁ aromatics, light hydrocarbons having 1-5 carbon atoms, and hydrogen. The reactor effluent stream is cooled with a high-pressure liquid stream from a high pressure product separator forming a cooled reactor effluent stream and a heated high pressure liquid stream. The cooled reactor effluent stream is separated in the high-pressure product separator forming a hydrogen stream and the high-pressure liquid stream. The heated high-pressure liquid stream is flashed in a first flash drum into a first flash drum vapor stream comprising the light hydrocarbons and a first flash drum liquid stream comprising the benzene, xylenes, toluene, and unconverted C₉-C₁₁ aromatics. The first flash drum liquid stream is divided into a first portion and a second portion. The first portion of the first flash drum liquid stream is recycled to the product condenser upstream of product high pressure separator. At least a part of the second portion of the first flash drum liquid stream is separated into a benzene stream, or a toluene stream, or a xylenes stream, or combinations thereof in an aromatics separation zone comprising a benzene-toluene column.

In some embodiments, the process further comprises: introducing the first flash drum vapor stream into a stripper column at a first position and introducing the second portion of the first flash liquid stream into the stripper column at a second position below the first position. The first flash drum vapor stream and the second portion of the first flash drum liquid stream are separated in the stripper column into a stripper column overhead stream comprising part of the benzene, non-aromatic compounds, and the light hydrocarbons and a stripper column bottom stream comprising the remaining part of benzene, xylenes, unconverted aromatic compounds, and toluene. In this case, separating at least the part of the second portion of the first flash drum liquid stream in the aromatics separation zone comprises separating the stripper column bottom stream in the aromatics separation zone.

In some embodiments, the process further comprises: precooling the reactor effluent stream with the combined feed stream before cooling the reactor effluent stream with the high-pressure liquid stream from the high pressure product separator forming a precooled reactor effluent stream. The precooled reactor effluent stream is separated in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator. The high pressure hot separator liquid stream is separated in a second flash drum into a second flash drum vapor stream and a second flash drum liquid stream. The second flash drum vapor stream is passed to the first flash drum. The second portion of the first flash drum liquid stream is passed to the benzene toluene column at a first position, and the second flash drum liquid stream is passed to the benzene-toluene column at a second position below the first position. The aromatics separation zone further comprises a benzene-toluene sidecut column. The process further comprises passing an aromatics-rich stream from a sulfolane extraction process to the benzene-toluene sidecut column, the aromatics-rich stream comprising benzene and toluene. The aromatics-rich stream from the extraction process is separated in the benzene-toluene sidecut column into the benzene stream, the toluene stream, or both. The benzene stream comprises an overhead stream or a sidecut stream from the benzene-toluene sidecut column at a position above where the aromatics-rich stream from the extraction process enters the benzene-toluene sidecut column. The toluene stream comprises a bottom stream from the benzene-toluene sidecut column, and the xylenes stream comprises a bottom stream from the benzene-toluene column.

In some embodiments, the process further comprises passing a sidecut stream from the benzene-toluene column to the benzene-toluene sidecut column at a position above a position where the aromatics-rich stream from the extraction process enters the benzene-toluene sidecut column. An overhead stream from the benzene-toluene sidecut column is passed to the benzene-toluene column at a position above a position where the sidecut stream exits the benzene-toluene column. The benzene stream exits the benzene-toluene sidecut column at a position below the position where the sidecut stream from the benzene-toluene column enters the benzene-toluene sidecut column and above the position where the aromatic stream from the extraction process enters the benzene-toluene sidecut column.

In some embodiments, the process further comprises dividing the hydrogen stream into a first portion and a second portion. The first portion of the hydrogen stream is compressed in a purge gas compressor forming a compressed first portion, and the compressed first portion of the hydrogen stream is separated in a membrane separation unit into a permeate stream comprising hydrogen and a hydrogen-depleted offgas stream. The second portion of the hydrogen stream is compressed in a recycle gas compressor forming a compressed second portion, the permeate stream and the compressed second portion are combined forming a combined hydrogen stream, and the combined hydrogen stream is recycled to the combined feed stream.

In some embodiments, the process further comprises precooling the reactor effluent stream with the combined feed stream before cooling the reactor effluent stream with the high-pressure liquid stream from the high-pressure product separator forming a precooled reactor effluent stream. The precooled reactor effluent stream is separated in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator. The hot separator liquid stream is flashed in a second flash drum into a second flash drum vapor stream and a second flash drum liquid stream. The second flash drum vapor stream is passed to the first flash drum. The second portion of the first flash drum liquid stream is passed to the benzene-toluene column at a first position, and the second flash drum liquid stream is passed to the benzene-toluene column at a second position below the first position. The aromatics separation zone further comprises a benzene-toluene sidecut column, and the process further comprises passing an aromatics-rich stream comprising benzene and toluene from a sulfolane extraction process to the benzene-toluene sidecut column. The aromatics-rich stream is separated in the benzene-toluene sidecut column into the benzene stream, the toluene column, or both. The benzene stream comprises an overhead stream or a sidecut stream from the benzene-toluene sidecut column at a position above where the aromatics-rich stream enters the benzene-toluene sidecut column, the toluene stream comprises a bottom stream from the benzene-toluene sidecut column, and the xylenes stream comprises a bottom stream from the benzene-toluene column.

In some embodiments, the process further comprises dividing the hydrogen stream into a first portion and a second portion. The first portion of the hydrogen stream is compressed in a purge gas compressor forming a compressed first portion. The compressed first portion of the hydrogen stream is separated in a membrane separation unit into a permeate stream comprising hydrogen and a hydrogen-depleted offgas stream. The second portion of the hydrogen stream is compressed in a recycle gas compressor forming a compressed second portion. The permeate stream and the compressed second portion of the hydrogen stream are combined forming a combined hydrogen stream, and the combined hydrogen stream is recycled to the combined feed stream.

In some embodiments the process further comprises separating a benzene-toluene column overhead stream into an overhead offgas stream comprising hydrogen, light hydrocarbons having 1-5 carbon atoms, and part of the benzene, and an overhead liquid stream comprising hydrogen, light hydrocarbons having 1-5 carbon atoms, and a second part of the benezene. The overhead offgas stream is compressed forming a compressed overhead offgas stream. The compressed overhead offgas stream and the overhead liquid stream are passed to the stabilizer column.

In some embodiments, the process further comprises compressing the hydrogen stream, and recycling the compressed hydrogen stream to the combined feed stream.

In some embodiments, the process further comprises preheating the combined feed stream in a heat exchanger with the reactor effluent stream, or a fired heater, or both before transalkylating the combined feed stream.

In some embodiments, the process comprises transalkylating a combined feed stream comprising toluene, C₉-C₁₁ aromatics, and hydrogen forming a reactor effluent stream comprising benzene, xylenes, toluene, unconverted C₉-C₁₁ aromatics, light hydrocarbons having 1-5 carbon atoms, and hydrogen. The reactor effluent stream is separated in a hot separator into a hot separator vapor stream and a hot separator liquid stream. The hot separator vapor stream is cooled with a high-pressure liquid stream from a high-pressure product separator forming a cooled hot separator vapor stream and a heated high-pressure liquid stream. The cooled hot separator vapor stream is separated in the high pressure product separator forming a hydrogen stream and the high-pressure liquid stream from the high-pressure product separator. The hot separator liquid stream is separated in a second flash drum into a second flash drum vapor stream and a second flash drum liquid stream. The heated high-pressure liquid stream and the second flash drum vapor stream are flashed in a first flash drum into a first flash drum vapor stream comprising the light hydrocarbons and a first flash drum liquid stream comprising the benzene, xylenes, and toluene. The first flash drum liquid stream is passed to an aromatics separation zone comprising a benzene-toluene column at a first position, the second flash drum liquid stream is passed to the benzene-toluene column at a second position below the first position. The first flash drum liquid stream and the second flash drum liquid stream are separated into a benzene stream, or a toluene stream, or a xylenes stream, or combinations thereof in the aromatics separation zone.

In some embodiments, the process further comprises precooling the reactor effluent stream with the combined feed stream before separating the reactor effluent stream in the hot separator forming a precooled reactor effluent stream. The reactor effluent stream separated in the hot separator comprises the precooled reactor effluent stream. The aromatics separation zone further comprises a benzene-toluene sidecut column, and the process further comprises passing an aromatics-rich stream comprising benzene and toluene from a sulfolane extraction process to the benzene-toluene sidecut column. The aromatics-rich stream is separated in the benzene-toluene sidecut column into the benzene stream, the toluene stream, or both. The benzene stream comprises an overhead stream or a sidecut stream from the benzene-toluene sidecut column at a position above where the aromatics-rich stream enters the benzene-toluene sidecut column, the toluene stream comprises a bottom stream from the benzene-toluene sidecut column, and the xylenes stream comprises a bottom stream from the benzene-toluene column.

In some embodiments, the process further comprises passing a sidecut stream from the benzene-toluene column to the benzene-toluene sidecut column at a position above a position where the aromatics-rich stream enters the benzene-toluene sidecut column, and passing an overhead stream from the benzene-toluene sidecut column to the benzene-toluene column at a position above a position where the sidecut stream exits the benzene-toluene column. The benzene stream exits the benzene-toluene sidecut column at a position below where the sidecut stream from the benzene-toluene column enters the benzene-toluene sidecut column.

In some embodiments, the process further comprises dividing the hydrogen stream into a first portion and a second portion. The first portion of the hydrogen stream is compressed in a purge gas compressor forming a compressed first portion, and the compressed first portion of the hydrogen stream is separated in a membrane separation unit into a permeate stream comprising hydrogen and a hydrogen depleted offgas stream. The second portion of the hydrogen stream is separated in a recycle gas compressor forming a compressed second portion. The permeate stream and the compressed second portion of the hydrogen stream are combined forming a combined hydrogen stream, and the combined hydrogen stream is recycled to the combined feed stream.

In some embodiments, the process comprises transalkylating a combined feed stream comprising toluene, C₉-C₁₁ aromatics, and hydrogen forming a reactor effluent stream comprising benzene, xylenes, toluene, unconverted aromatic compounds having 9-11 carbon atoms, light hydrocarbons having 1-5 carbon atoms, and hydrogen. The reactor effluent stream is cooled with a high pressure separator liquid stream from a high pressure product separator forming a cooled reactor effluent stream and a heated high pressure separator liquid stream. The cooled reactor effluent stream is separated in the high pressure product separator forming a hydrogen stream and the high pressure separator liquid stream. The heated high pressure liquid stream is heated with a stripper sidecut stream from a stripper column forming a second heated high pressure liquid stream and a cooled sidecut stream. The second heated high pressure liquid stream is separated in the stripper column into a stripper overhead stream comprising part of the benzene, non-aromatic compounds, and the light hydrocarbons, the stripper sidecut stream comprising benzene, xylenes, unconverted C₉-C₁₁ aromatic compounds, and toluene, and a stripper bottom stream comprising the benzene, xylenes, unconverted C₉-C₁₁ aromatic compounds, and toluene. The second heated high pressure stream enters the stripper column at a position above where the sidecut stream exits the stripper column. The cooled stripper sidecut stream is combined with the cooled reactor effluent stream. The stripper bottom stream is separated into a benzene stream, or a toluene stream, or a xylenes stream, or combinations thereof in an aromatics separation zone.

In some embodiments, the process further comprises heating the second heated liquid stream with the stripper bottom stream before separating the second heated high pressure liquid stream in the stripper column.

In some embodiments, the process further comprises compressing the hydrogen stream, and recycling the compressed hydrogen stream to the combined feed stream.

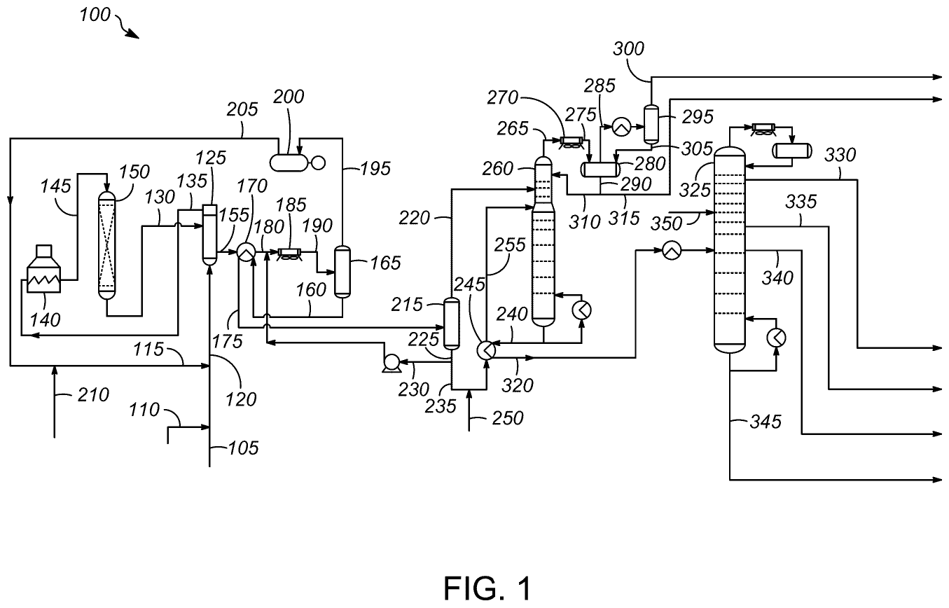

FIG. 1 illustrates an aromatic transalkylation process 100. The C₉-C₁₁ aromatics stream 105, toluene stream 110, and hydrogen stream 115 are combined into combined feed stream 120. The combined feed stream 120 is sent to combined feed heat exchanger 125 where it is heated with the reactor effluent stream 130. The heated combined feed stream 135 is further heated in a charge heater 140, and the heated combined feed stream 145 is sent to aromatic transalkylation reactor 150.

The reactor effluent stream 130, which comprises benzene, toluene, xylenes, light hydrocarbons (e.g., C₁ to C₅ hydrocarbons), is cooled in the combined feed heat exchanger 125. The cooled reactor effluent stream 155 is heat exchanged with the high-pressure bottom liquid stream 160 from the high-pressure product separator 165 in heat exchanger 170 forming heated high-pressure liquid stream 175 and cooled reactor effluent stream 180.

The cooled reactor effluent stream 180 is condensed in product condenser 185, and the condensed reactor effluent stream 190 is sent to the high-pressure product separator 165 where it is separated into an overhead gas stream 195 and high-pressure bottom liquid stream 160. The overhead gas stream 195 comprising hydrogen is compressed in compressor 200 forming compressed hydrogen stream 205. The compressed hydrogen stream 205 is combined with makeup hydrogen stream 210 to form the hydrogen stream 115.

The heated high-pressure liquid stream 175 is sent to flash drum 215 where it is flashed into a flash drum vapor stream 220 comprising the C₁ to C₅ light hydrocarbons and a flash drum bottom liquid stream 225 comprising the benzene, toluene, and xylenes.

A portion 230 of the flash drum bottom liquid stream 225 is combined with the cooled reactor effluent stream 180 and sent to the product condenser 185. Another portion 235 of the flash drum bottom liquid stream 225 is heat exchanged with the stripper bottom stream 240 in heat exchanger 245. Optionally, an A8 stripper column overhead stream 250 can be combined with the portion 235 of the flash drum bottom liquid stream 225.

The heated flash drum bottom liquid stream 255 and the flash drum vapor stream 220 are sent to stripper column 260 and separated into stripper overhead stream 265 and stripper bottom stream 240.

The stripper overhead stream 265 is condensed in overhead condenser 270, and the condensed stream 275 is separated in overhead separator 280 into an overhead vapor stream 285 and an overhead liquid stream 290. The overhead vapor stream 285 is separated in a separator 295 into an offgas stream 300 and a liquid stream 305. Offgas stream 300 is sent to a fuel gas header. Liquid stream 305 is sent to the overhead separator 280.

The overhead liquid stream 290 from the overhead separator 280 is divided into portion 310 which is refluxed to the stripper column 260 and portion 315 which is sent to a stabilizer column.

The cooled stripper bottom stream 320 from the heat exchanger 245 is sent to BT column 325 where it is separated into benzene stream 330, toluene stream 335, toluene stream (makeup desorbent) 340, and xylenes stream 345.

Optionally, a stream 350 comprising benzene and toluene from an extraction process can be sent to the BT column 325.

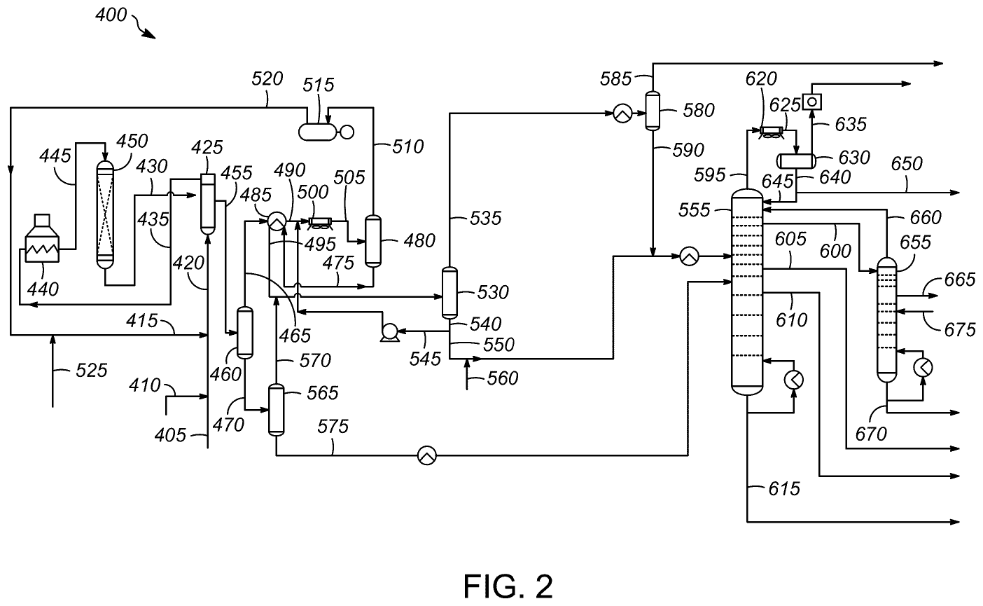

FIG. 2 illustrates an aromatic transalkylation process 400. The C₉-C₁₁ aromatics stream 405, toluene stream 410, and hydrogen stream 415 are combined into combined feed stream 420. The combined feed stream 420 is sent to combined feed heat exchanger 425 where it is heated with the reactor effluent stream 430. The heated combined feed stream 435 is further heated in a charge heater 440, and the heated combined feed stream 445 is sent to aromatic transalkylation reactor 450.

The reactor effluent stream 430, which comprises benzene, toluene, xylenes, light hydrocarbons (e.g., C₁ to C₅ hydrocarbons), is cooled in the combined feed heat exchanger 425. The cooled reactor effluent stream 455 is separated in a hot separator 460 into hot separator vapor stream 465 and hot separator liquid stream 470.

The hot separator vapor stream 465 is heat exchanged with the high-pressure bottom liquid stream 475 from the high-pressure product separator 480 in heat exchanger 485 forming cooled hot separator vapor stream 490 and heated high-pressure bottom liquid stream 495. The cooled hot separator vapor stream 490 is condensed in product condenser 500, and the condensed hot separator vapor stream 505 is sent to the high-pressure product separator 480. The condensed hot separator vapor stream 505 is separated into an overhead gas stream 510 and high-pressure bottom liquid stream 475. The overhead gas stream 510 comprising hydrogen is compressed in compressor 515 forming compressed hydrogen stream 520. The compressed hydrogen stream 520 is combined with makeup hydrogen stream 525 to form the hydrogen stream 415.

The heated high-pressure bottom liquid stream 495 is sent to the first flash drum 530 where it is flashed into a first flash drum overhead stream 535 comprising the C₁ to C₅ light hydrocarbons and a first flash drum bottom liquid stream 540 comprising the benzene, toluene, and xylenes.

A portion 545 of the first flash drum bottom liquid stream 540 is combined with the cooled hot separator vapor stream 490 and sent to the product condenser 500. Another portion 550 of the first flash drum bottom liquid stream 540 is sent to BT column 555. Optionally, an A8 stripper column overhead stream 560 can be combined with the portion 550 of the first flash drum bottom liquid stream 540.

The hot separator liquid stream 470 is sent to a second flash drum 565 where it is separated into a second flash drum vapor stream 570 and a second flash drum liquid stream 575. The second flash drum vapor stream 570 is combined with the heated high-pressure bottom liquid stream 495 and sent to the first flash drum 530. The second flash drum liquid stream 575 is sent to the BT column 555 at a position below the position where the portion 550 of the first flash drum bottom liquid stream 540 enters.

The first flash drum overhead stream 535 is sent to a separator 580 and separated into an offgas stream 585 and a liquid stream 590. The offgas stream 585 can be sent to a fuel gas header. The liquid stream 590 can be combined with the portion 550 of the first flash drum bottom liquid stream 540 and sent to the BT column 555.

The portion 550 of the first flash drum bottom liquid stream 540 and the second flash drum liquid stream 575 are separated in the BT column 555 into an overhead stream 595 comprising C₁-C₅ hydrocarbons, a sidecut stream 600 comprising benzene and toluene, a toluene stream 605, a toluene stream (makeup desorbent) 610, and xylenes stream 615.

The overhead stream 595 is condensed in condenser 620. The condensed overhead stream 625 is separated in overhead separator 630 into an off-gas stream 635 and an overhead liquid stream 640. The off-gas stream 635 can be sent to a fuel gas header, The overhead liquid stream 640 is divided into a portion 645 which is refluxed to the BT column 555 and a portion 650 which is sent to the stabilizer column.

The sidecut stream 600 is sent to a BT sidecut column 655 where it is separated into an overhead stream 660 comprising benzene and residual non-aromatics, benzene stream 665, and toluene stream 670. The overhead stream 660 is returned to the BT column 555 at a point above where sidecut stream 600 exited. Optionally, a stream 675 comprising benzene and toluene from an extraction process can be sent to the BT sidecut column 655.

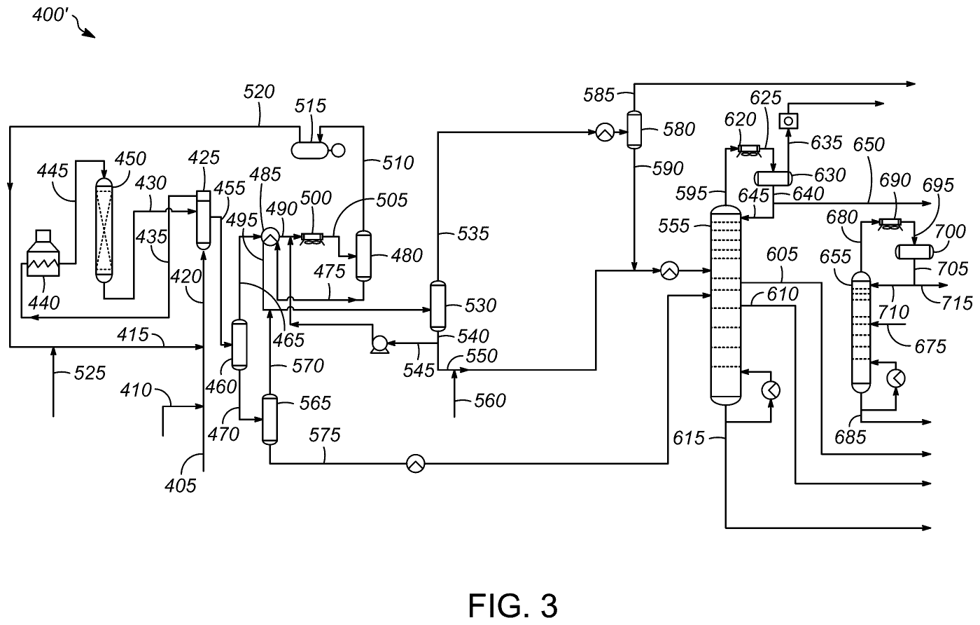

FIG. 3 illustrates an aromatic transalkylation process 400′. The C₉-C₁₁ aromatics stream 405, toluene stream 410, and hydrogen stream 415 are combined into combined feed stream 420. The combined feed stream 420 is sent to combined feed heat exchanger 425 where it is heated with the reactor effluent stream 430. The heated combined feed stream 435 is further heated in a charge heater 440, and the heated combined feed stream 445 is sent to aromatic transalkylation reactor 450.

The reactor effluent stream 430, which comprises benzene, toluene, xylenes, light hydrocarbons (e.g., C₁ to C₅ hydrocarbons), is cooled in the combined feed heat exchanger 425. The cooled reactor effluent stream 455 is separated in a hot separator 460 into hot separator vapor stream 465 and hot separator liquid stream 470.

The hot separator vapor stream 465 is heat exchanged with the high-pressure bottom liquid stream 475 from the high-pressure product separator 480 in heat exchanger 485 forming cooled hot separator vapor stream 490 and heated high-pressure bottom liquid stream 495. The cooled hot separator vapor stream 490 is condensed in product condenser 500, and the condensed hot separator vapor stream 505 is sent to the high-pressure product separator 480. The condensed hot separator vapor stream 505 is separated into an overhead gas stream 510 and high-pressure bottom liquid stream 475. The overhead gas stream 510 comprising hydrogen is compressed in compressor 515 forming compressed hydrogen stream 520. The compressed hydrogen stream 520 is combined with makeup hydrogen stream 525 to form the hydrogen stream 415.

The heated high-pressure bottom liquid stream 495 is sent to the first flash drum 530 where it is flashed into a first flash drum overhead stream 535 comprising the C₁ to C₅ light hydrocarbons and a first flash drum bottom liquid stream 540 comprising the benzene, toluene, and xylenes.

A portion 545 of the first flash drum bottom liquid stream 540 is combined with the cooled hot separator vapor stream 490 and sent to the product condenser 500. Another portion 550 of the first flash drum bottom liquid stream 540 is sent to BT column 555. Optionally, an A8 stripper column overhead stream 560 can be combined with the portion 550 of the first flash drum bottom liquid stream 540.

The hot separator liquid stream 470 is sent to a second flash drum 565 where it is separated into a second flash drum vapor stream 570 and a second flash drum liquid stream 575. The second flash drum vapor stream 570 is combined with the heated high-pressure bottom liquid stream 495 and sent to the first flash drum 530. The second flash drum liquid stream 575 is sent to the BT column 555 at a position below the position where the portion 550 of the first flash drum bottom liquid stream 540 enters.

The first flash drum overhead stream 535 is sent to a separator 580 and separated into an offgas stream 585 and a liquid stream 590. The offgas stream 585 can be sent to a fuel gas header. The liquid stream 590 can be combined with the portion 550 of the first flash drum bottom liquid stream 540 and sent to the BT column 555.

The portion 550 of the first flash drum bottom liquid stream 540 and the second flash drum liquid stream 575 are separated in the BT column into an overhead stream 595 comprising benzene, a toluene stream 605, a toluene stream (makeup desorbent) 610, and xylenes stream 615.

The overhead stream 595 is condensed in condenser 620. The condensed overhead stream 625 is separated in overhead separator 630 into an off-gas stream 635 and an overhead liquid stream 640. The off-gas stream 635 can be sent to a fuel gas header. The overhead liquid stream 640 is divided into a portion 645 which is refluxed to the BT column 555 and a portion 650 which is sent to the stabilizer column.

The BT sidecut column 655 is separate from the BT column 555. A stream 675 comprising benzene and toluene from an extraction process can be sent to the BT sidecut column 655 where it is separated into an overhead stream 680 comprising benzene and bottom stream 685 comprising toluene. The overhead stream 680 is condensed in condenser 690. The condensed overhead stream 695 is sent to an overhead receiver 700. The liquid stream 705 from the overhead receiver 700 is divided into a portion 710 which is refluxed to the BT sidecut column 655 and benzene stream 715 which is recovered as benzene product. Alternatively, benzene stream 715 can also be drawn from the BT sidecut column 655.

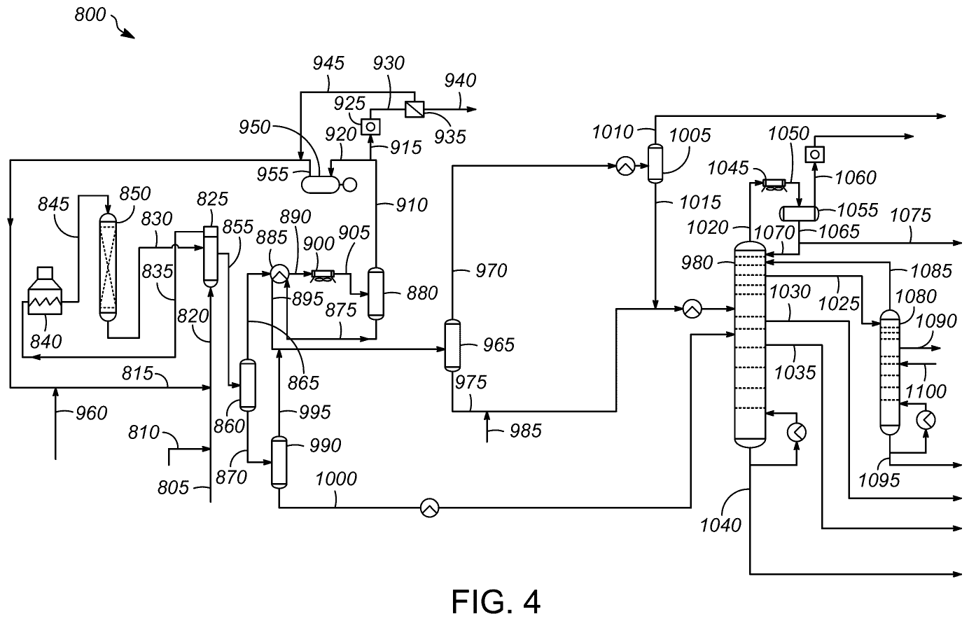

FIG. 4 illustrates an aromatic transalkylation process 800. The C₉-C₁₁ aromatics stream 805, toluene stream 810, and hydrogen stream 815 are combined into combined feed stream 820. The combined feed stream 820 is sent to combined feed heat exchanger 825 where it is heated with the reactor effluent stream 830. The heated combined feed stream 835 is further heated in a charge heater 840, and the heated combined feed stream 845 is sent to aromatic transalkylation reactor 850.

The reactor effluent stream 830, which comprises benzene, toluene, xylenes, light hydrocarbons (e.g., C₁ to C₅ hydrocarbons), is cooled in the combined feed heat exchanger 825. The cooled reactor effluent stream 855 is separated in a hot separator 860 into hot separator vapor stream 865 and hot separator liquid stream 870.

The hot separator vapor stream 865 is heat exchanged with the high-pressure bottom liquid stream 875 from the high-pressure product separator 880 in heat exchanger 885 forming cooled hot separator vapor stream 890 and heated high-pressure bottom liquid stream 895. The cooled hot separator vapor stream 890 is condensed in product condenser 900, and the condensed hot separator vapor stream 905 is sent to the high-pressure product separator 880. The condensed hot separator vapor stream 905 is separated into an overhead gas stream 910 and high-pressure bottom liquid stream 875.

The overhead gas stream 910 comprising hydrogen is divided into a first portion 915 and a second portion 920. The first portion 915 is compressed in compressor 925, and the compressed first portion 930 is sent to a membrane separation unit 935 where it is separated into an offgas stream 940 comprising light hydrocarbons (e.g., C₁ to C₅ hydrocarbons), and a permeate stream 945 comprising hydrogen.

The second portion 920 of the overhead gas stream 910 is compressed in compressor 950 forming compressed hydrogen stream 955. The compressed hydrogen stream 955 is combined with permeate stream 945 and makeup hydrogen stream 960 to form the hydrogen stream 815.

The heated high-pressure bottom liquid stream 895 is sent to the first flash drum 965 where it is flashed into a first flash drum overhead stream 970 comprising the C₁ to C₅ light hydrocarbons and a first flash drum bottom liquid stream 975 comprising the benzene, toluene, and xylenes.

The first flash drum bottom liquid stream 975 is sent to BT column 980. Optionally, an A8 stripper column overhead stream 985 can be combined with the first flash drum bottom liquid stream 975.

The hot separator liquid stream 870 is sent to a second flash drum 990 where it is separated into a second flash drum vapor stream 995 and a second flash drum liquid stream 1000. The second flash drum vapor stream 995 is combined with the heated high-pressure bottom liquid stream 895 and sent to the first flash drum 965. The second flash drum liquid stream 1000 is sent to the BT column 980 at a position below the position where the first flash drum bottom liquid stream 975 enters.

The first flash drum overhead stream 970 is sent to a separator 1005 and separated into an offgas stream 1010 and a liquid stream 1015. The offgas stream 1010 can be sent to a fuel gas header. The liquid stream 1015 can be combined with the first flash drum bottom liquid stream 975 and sent to the BT column 980.

The first flash drum bottom liquid stream 975 and the second flash drum liquid stream 1000 are separated in the BT column 980 into an overhead stream 1020 comprising benzene, a sidecut stream 1025 comprising benzene and toluene, a toluene stream 1030, a toluene stream (makeup desorbent) 1035, and xylenes stream 1040.

The overhead stream 1020 is condensed in condenser 1045. The condensed overhead stream 1050 is separated in overhead separator 1055 into an off gas stream 1060 and an overhead liquid stream 1065. The off gas stream 1060 can be sent to a fuel gas header. The overhead liquid stream 1065 is divided into a portion 1070 which is refluxed to the BT column 980 and a portion 1075 which is sent to the stabilizer column.

The sidecut stream 1025 is sent to a BT sidecut column 1080 where it is separated into an overhead stream 1085 comprising benzene and residual non-aromatics, benzene stream 1090, and toluene stream 1095. The overhead stream 1085 is returned to the BT column 980 at a point above where the sidecut stream 1025 exited. Optionally, a stream 1100 comprising benzene and toluene from an extraction process can be sent to the BT sidecut column 1080.

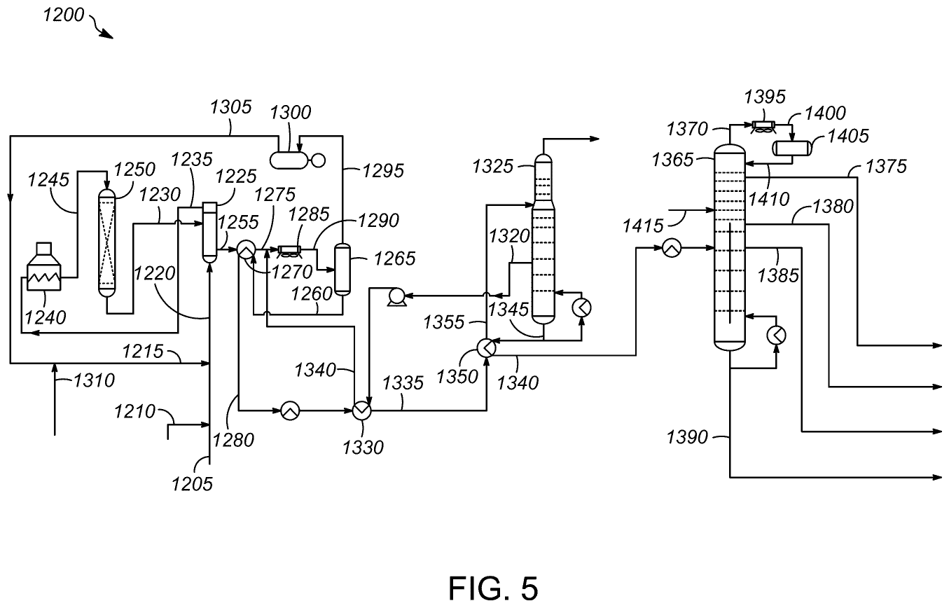

FIG. 5 illustrates an aromatic transalkylation process 1200. The C₉-C₁₁ aromatics stream 1205, toluene stream 1210, and hydrogen stream 1215 are combined into combined feed stream 1220. The combined feed stream 1220 is sent to combined feed heat exchanger 1225 where it is heated with the reactor effluent stream 1230. The heated combined feed stream 1235 is further heated in a charge heater 1240, and the heated combined feed stream 1245 is sent to aromatic transalkylation reactor 1250.

The reactor effluent stream 1230, which comprises benzene, toluene, xylenes, light hydrocarbons (e.g., C₁ to C₅ hydrocarbons), is cooled in the combined feed heat exchanger 1225.

The cooled reactor effluent stream 1255 is heat exchanged with the high-pressure bottom liquid stream 1260 from the high-pressure product separator 1265 in heat exchanger 1270 forming cooled reactor effluent stream 1275 and heated high-pressure bottom liquid stream 1280. The cooled reactor effluent stream 1275 is condensed in product condenser 1285, and the condensed reactor effluent stream 1290 is sent to the high-pressure product separator 1265. The condensed reactor effluent stream 1290 is separated into an overhead gas stream 1295 comprising hydrogen and high-pressure bottom liquid stream 1260.

The overhead gas stream 1295 comprising hydrogen is compressed in compressor 1300 forming compressed hydrogen stream 1305. The compressed hydrogen stream 1305 is combined with makeup hydrogen stream 1310 to form the hydrogen stream 1215.

The heated high-pressure bottom liquid stream 1280 is heat exchanged with a stripper side cut stream 1320 from a stripper column 1325 in heat exchanger 1330 forming second heated high-pressure bottom liquid stream 1335 and cooled stripper side cut stream 1340. The cooled stripper side cut stream 1340 is combined with cooled reactor effluent stream 1275 and sent to product condenser 1285.

The second heated high-pressure bottom liquid stream 1335 is heat exchanged with the stripper bottom stream 1345 from the stripper column 1325 in heat exchanger 1350. The third heated high-pressure bottom liquid stream 1355 is sent to the stripper column 1325 at a position above where the stripper side cut stream 1320 exited.

The cooled stripper bottom stream 1360 is sent to the BT column 1365 where it is separated into an overhead stream 1370 comprising benzene, a benzene stream 1375, a toluene stream 1380, a toluene stream (makeup desorbent) 1385, and xylenes stream 1390.

The overhead stream 1370 is condensed in condenser 1395. The condensed overhead stream 1400 sent to a receiver 1405, and the overhead liquid stream 1410 is refluxed to the BT column 1365.

A stream 1415 comprising benzene and toluene from an extraction process can be sent to the BT column 1365.

### EXAMPLE

The flowscheme improvements resulted in major energy savings compared to a base case design without these changes. A commercial aromatics complex design with 1000 KMTA p-xylene production capacity was evaluated using commercially available process modeling software (UniSim™ Design Suite available from Honeywell). The commercial complex with base case contains a combined heat exchanger, charge heater, a reactor for catalytic disproportionation and transalkylation, a condenser, a product separator, a stripper column, and a benzene-toluene column. The feed to the commercial aromatics complex was a typical reformate feed stream with a RON of 106. Using the process shown in FIG. 2 with a feed of toluene and C₉₋₁₁ aromatics, the total reboiler duty was reduced by 72 MMBtu/hr from the base case requirement with a reduction in the stripper reboiler duty. This represents a 15% reduction in the total fired heater duty requirement for the aromatics complex, as shown in Table 1. The results would be similar for the processes shown in FIGS. 3-4. An equivalent reduction in condensing duty (air cooled) compared to base case is expected. In addition, there are hydrogen savings for all of the processes shown in FIGS. 1-5.

TABLE 1

| Description | Units | Base Case | Improved Case |
|---|---|---|---|
| Stripper Column | | | |
| Stripper Reboiler duty | MMBTU/hr | 68 | 0 |
| BT Column | | | |
| Reboiler duty | MMBTU/hr | 144 | 140 |
| Sidecut Reboiler duty | MMBTU/hr | 65 | 35 |
| BT Sidecut Column (New) | | | |
| Reboiler duty | MMBTU/hr | — | 30 |
| Total Reboiler duty | MMBTU/hr | 277 | 205 |
| Reduction in Reboiler duty | MMBTU/hr | — | 72 |

### SPECIFIC EMBODIMENTS

While the following is described in conjunction with specific embodiments, it will be understood that this description is intended to illustrate and not limit the scope of the preceding description and the appended claims.

A first embodiment of the invention is a process comprising transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen; cooling the reactor effluent stream with a high-pressure liquid stream from a high pressure product separator forming a cooled reactor effluent stream and a heated high pressure liquid stream; separating the cooled reactor effluent stream in the high-pressure product separator forming a hydrogen stream and the high-pressure liquid stream; flashing the heated high-pressure liquid stream in a first flash drum into a first flash drum vapor stream comprising the light hydrocarbons and a first flash drum liquid stream comprising the benzene, xylenes, toluene, and unconverted aromatic compounds having 9-11 carbon atoms; dividing the first flash drum liquid stream into a first portion and a second portion; recycling the first portion of the first flash drum liquid stream to the product condenser upstream of product high pressure separator; separating at least a part of the second portion of the first flash drum liquid stream into a benzene stream, or a toluene stream, or a xylenes stream, or combinations thereof in an aromatics separation zone comprising a benzene-toluene column. An embodiment of the invention is one, any or all of prior embodiments in this paragraph up through the first embodiment in this paragraph further comprising introducing the first flash drum vapor stream into a stripper column at a first position and introducing the second portion of the first flash liquid stream into the stripper column at a second position below the first position; separating the first flash drum vapor stream and the second portion of the first flash drum liquid stream in the stripper column into a stripper column overhead stream comprising part of the benzene, non-aromatic compounds, and the light hydrocarbons and a stripper column bottom stream comprising the remaining part of benzene, xylenes, unconverted aromatic compounds having 9-11 carbon atoms, and toluene; and wherein separating at least the part of the second portion of the first flash drum liquid stream in the aromatics separation zone comprises separating the stripper column bottom stream in the aromatics separation zone. An embodiment of the invention is one, any or all of prior embodiments in this paragraph up through the first embodiment in this paragraph further comprising precooling the reactor effluent stream with the combined feed stream before cooling the reactor effluent stream with the high-pressure liquid stream from the high pressure product separator forming a precooled reactor effluent stream; separating the precooled reactor effluent stream in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator; flashing the hot separator liquid stream in a second flash drum into a second flash drum vapor stream and a second flash drum liquid stream; passing the second flash drum vapor stream to the first flash drum; passing the second portion of the first flash drum liquid stream to the benzene toluene column at a first position; passing the second flash drum liquid stream to the benzene-toluene column at a second position below the first position; wherein the aromatics separation zone further comprises a benzene-toluene sidecut column, and further comprising passing an aromatics-rich stream from a sulfolane extraction process to the benzene-toluene sidecut column, the aromatics-rich stream comprising benzene and toluene; separating the aromatics-rich stream from the extraction process in the benzene-toluene sidecut column into the benzene stream, the toluene stream, or both; wherein the benzene stream comprises an overhead stream or a sidecut stream from the benzene-toluene sidecut column at a position above where the aromatics-rich stream from the extraction process enters the benzene-toluene sidecut column; wherein the toluene stream comprises a bottom stream from the benzene-toluene sidecut column; and wherein the xylenes stream comprises a bottom stream from the benzene-toluene column. An embodiment of the invention is one, any or all of prior embodiments in this paragraph up through the first embodiment in this paragraph further comprising passing a sidecut stream from the benzene-toluene column to the benzene-toluene sidecut column at a position above a position where the aromatics-rich stream from the extraction process enters the benzene-toluene sidecut column; and passing an overhead stream from the benzene-toluene sidecut column to the benzene-toluene column at a position above a position where the sidecut stream exits the benzene-toluene column; wherein the benzene stream exits the benzene-toluene sidecut column at a position below the position where the sidecut stream from the benzene-toluene column enters the benzene-toluene sidecut column and above the position where the aromatic stream from the extraction process enters the benzene-toluene sidecut column. An embodiment of the invention is one, any or all of prior embodiments in this paragraph up through the first embodiment in this paragraph further comprising dividing the hydrogen stream into a first portion and a second portion; compressing the first portion of the hydrogen stream in a purge gas compressor forming a compressed first portion; separating the compressed first portion of the hydrogen stream in a membrane separation unit into a permeate stream comprising hydrogen and a hydrogen-depleted offgas stream; compressing the second portion of the hydrogen stream in a recycle gas compressor forming a compressed second portion; combining the permeate stream and the compressed second portion forming a combined hydrogen stream; and recycling the combined hydrogen stream to the combined feed stream. An embodiment of the invention is one, any or all of prior embodiments in this paragraph up through the first embodiment in this paragraph further comprising precooling the reactor effluent stream with the combined feed stream before cooling the reactor effluent stream with the high-pressure liquid stream from the high-pressure product separator forming a precooled reactor effluent stream; separating the precooled reactor effluent stream in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator; flashing the hot separator liquid stream in a second flash drum into a second flash drum vapor stream and a second flash drum liquid stream; passing the second flash drum vapor stream to the first flash drum; passing the second portion of the first flash drum liquid stream to the benzene-toluene column at a first position; passing the second flash drum liquid stream to the benzene-toluene column at a second position below the first position; wherein the aromatics separation zone further comprises a benzene-toluene sidecut column, and further comprising passing an aromatics-rich stream comprising benzene and toluene from a sulfolane extraction process to the benzene-toluene sidecut column; separating the aromatics-rich stream in the benzene-toluene sidecut column into the benzene stream, the toluene column, or both; wherein the benzene stream comprises an overhead stream or a sidecut stream from the benzene-toluene sidecut column at a position above where the aromatics-rich stream enters the benzene-toluene sidecut column; wherein the toluene stream comprises a bottom stream from the benzene-toluene sidecut column; and wherein the xylenes stream comprises a bottom stream from the benzene-toluene column. An embodiment of the invention is one, any or all of prior embodiments in this paragraph up through the first embodiment in this paragraph further comprising dividing the hydrogen stream into a first portion and a second portion; compressing the first portion of the hydrogen stream in a purge gas compressor forming a compressed first portion; separating the compressed first portion of the hydrogen stream in a membrane separation unit into a permeate stream comprising hydrogen and a hydrogen-depleted offgas stream; compressing the second portion of the hydrogen stream in a recycle gas compressor forming a compressed second portion; combining the permeate stream and the compressed second portion of the hydrogen stream forming a combined hydrogen stream; and recycling the combined hydrogen stream to the combined feed stream. An embodiment of the invention is one, any or all of prior embodiments in this paragraph up through the first embodiment in this paragraph further comprising separating a benzene-toluene column overhead stream into an overhead offgas stream comprising hydrogen, light hydrocarbons having 1-5 carbon atoms, and part of the benzene, and an overhead liquid stream comprising hydrogen, light hydrocarbons having 1-5 carbon atoms, and a second part of the benzene; compressing the overhead offgas stream forming a compressed overhead offgas stream; passing the compressed overhead offgas stream to a stabilizer column passing the overhead liquid stream to the stabilizer column. An embodiment of the invention is one, any or all of prior embodiments in this paragraph up through the first embodiment in this paragraph further comprising compressing the hydrogen stream; and recycling the compressed hydrogen stream to the combined feed stream. An embodiment of the invention is one, any or all of prior embodiments in this paragraph up through the first embodiment in this paragraph further comprising preheating the combined feed stream in a heat exchanger with the reactor effluent stream, or a fired heater, or both before transalkylating the combined feed stream.

A second embodiment of the invention is a process comprising transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen; separating the reactor effluent stream in a hot separator into a hot separator vapor stream and a hot separator liquid stream; cooling the hot separator vapor stream with a high-pressure liquid stream from a high-pressure product separator forming a cooled hot separator vapor stream and a heated high-pressure liquid stream; separating the cooled hot separator vapor stream in the high pressure product separator forming a hydrogen stream and the high-pressure liquid stream from the high-pressure product separator; flashing the hot separator liquid stream in a second flash drum into a second flash drum vapor stream and a second flash drum liquid stream; flashing the heated high-pressure liquid stream and the second flash drum vapor stream in a first flash drum into a first flash drum vapor stream comprising the light hydrocarbons and a first flash drum liquid stream comprising the benzene, the xylenes, and the toluene; passing the first flash drum liquid stream to an aromatics separation zone comprising a benzene-toluene column at a first position; passing the second flash drum liquid stream to the benzene-toluene column at a second position below the first position; separating the first flash drum liquid stream and the second flash drum liquid stream into a benzene stream, or a toluene stream, or a xylenes stream, or combinations thereof in the aromatics separation zone. An embodiment of the invention is one, any or all of prior embodiments in this paragraph up through the second embodiment in this paragraph further comprising precooling the reactor effluent stream with the combined feed stream before separating the reactor effluent stream in the hot separator forming a precooled reactor effluent stream; wherein separating the reactor effluent stream in the hot separator comprises separating the precooled reactor effluent stream in the hot separator; wherein the aromatics separation zone further comprises a benzene-toluene sidecut column, and further comprising passing an aromatics-rich stream comprising benzene and toluene from a sulfolane extraction process to the benzene-toluene sidecut column; separating the aromatics-rich stream in the benzene-toluene sidecut column into the benzene stream, the toluene stream, or both; wherein the benzene stream comprises an overhead stream or a sidecut stream from the benzene-toluene sidecut column at a position above where the aromatics-rich stream enters the benzene-toluene sidecut column; wherein the toluene stream comprises a bottom stream from the benzene-toluene sidecut column; and wherein the xylenes stream comprises a bottom stream from the benzene-toluene column. An embodiment of the invention is one, any or all of prior embodiments in this paragraph up through the second embodiment in this paragraph further comprising passing a sidecut stream from the benzene-toluene column to the benzene-toluene sidecut column at a position above a position where the aromatics-rich stream enters the benzene-toluene sidecut column; and passing an overhead stream from the benzene-toluene sidecut column to the benzene-toluene column at a position above a position where the sidecut stream exits the benzene-toluene column; wherein the benzene stream exits the benzene-toluene sidecut column at a position below where the sidecut stream from the benzene-toluene column enters the benzene-toluene sidecut column. An embodiment of the invention is one, any or all of prior embodiments in this paragraph up through the second embodiment in this paragraph further comprising dividing the hydrogen stream into a first portion and a second portion; compressing the first portion of the hydrogen stream in a purge gas compressor forming a compressed first portion; separating the compressed first portion of the hydrogen stream in a membrane separation unit into a permeate stream comprising hydrogen and a hydrogen depleted offgas stream; compressing the second portion of the hydrogen stream in a recycle gas compressor forming a compressed second portion; combining the permeate stream and the compressed second portion of the hydrogen stream forming a combined hydrogen stream; and recycling the combined hydrogen stream to the combined feed stream. An embodiment of the invention is one, any or all of prior embodiments in this paragraph up through the second embodiment in this paragraph further comprising compressing the hydrogen stream; and recycling the compressed hydrogen stream to the combined feed stream. An embodiment of the invention is one, any or all of prior embodiments in this paragraph up through the second embodiment in this paragraph further comprising preheating the combined feed stream in a heat exchanger with the reactor effluent stream, or a fired heater, or both before transalkylating the combined feed stream.

A third embodiment of the invention is a process comprising transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen; cooling the reactor effluent stream with a high pressure separator liquid stream from a high pressure product separator forming a cooled reactor effluent stream and a heated high pressure separator liquid stream; separating the cooled reactor effluent stream in the high pressure product separator forming a hydrogen stream and the high pressure separator liquid stream; heating the heated high pressure liquid stream with a stripper sidecut stream from a stripper column forming a second heated high pressure liquid stream and a cooled sidecut stream; separating the second heated high pressure liquid stream in the stripper column into a stripper overhead stream comprising part of the benzene, non-aromatic compounds, and the light hydrocarbons, the stripper sidecut stream comprising benzene, xylenes, unconverted aromatic compounds, and toluene, and a stripper bottom stream comprising the benzene, the xylenes, the unconverted aromatic compounds, and the toluene, wherein the second heated high pressure stream enters the stripper column at a position above where the sidecut stream exits the stripper column; combining the cooled stripper sidecut stream with the cooled reactor effluent stream; and separating the stripper bottom stream into a benzene stream, or a toluene stream, or a xylenes stream, or combinations thereof in an aromatics separation zone. An embodiment of the invention is one, any or all of prior embodiments in this paragraph up through the third embodiment in this paragraph further comprising heating the second heated liquid stream with the stripper bottom stream before separating the second heated high pressure liquid stream in the stripper column. An embodiment of the invention is one, any or all of prior embodiments in this paragraph up through the third embodiment in this paragraph further comprising compressing the hydrogen stream; and recycling the compressed hydrogen stream to the combined feed stream. An embodiment of the invention is one, any or all of prior embodiments in this paragraph up through the third embodiment in this paragraph further comprising preheating the combined feed stream in a heat exchanger with the reactor effluent stream, or a fired heater, or both before transalkylating the combined feed stream.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process comprising:
transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen;
cooling the reactor effluent stream with a high-pressure liquid stream from a high pressure product separator forming a cooled reactor effluent stream and a heated high pressure liquid stream;
separating the cooled reactor effluent stream in the high-pressure product separator forming a hydrogen stream and the high-pressure liquid stream;
flashing the heated high-pressure liquid stream in a first flash drum into a first flash drum vapor stream comprising the light hydrocarbons and a first flash drum liquid stream comprising the benzene, xylenes, toluene, and unconverted aromatic compounds having 9-11 carbon atoms;
dividing the first flash drum liquid stream into a first portion and a second portion;
recycling the first portion of the first flash drum liquid stream to the product condenser upstream of product high pressure separator; and
separating at least a part of the second portion of the first flash drum liquid stream into a benzene stream, or a toluene stream, or a xylenes stream, or combinations thereof in an aromatics separation zone comprising a benzene-toluene column.

2. The process of claim 1 further comprising:
introducing the first flash drum vapor stream into a stripper column at a first position and introducing the second portion of the first flash liquid stream into the stripper column at a second position below the first position;
separating the first flash drum vapor stream and the second portion of the first flash drum liquid stream in the stripper column into a stripper column overhead stream comprising part of the benzene, non-aromatic compounds, and the light hydrocarbons and a stripper column bottom stream comprising the remaining part of benzene, xylenes, unconverted aromatic compounds having 9-11 carbon atoms, and toluene; and
wherein separating at least the part of the second portion of the first flash drum liquid stream in the aromatics separation zone comprises separating the stripper column bottom stream in the aromatics separation zone.

3. The process of claim 1 further comprising:
precooling the reactor effluent stream with the combined feed stream before cooling the reactor effluent stream with the high-pressure liquid stream from the high pressure product separator forming a precooled reactor effluent stream;
separating the precooled reactor effluent stream in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator;
flashing the hot separator liquid stream in a second flash drum into a second flash drum vapor stream and a second flash drum liquid stream;
passing the second flash drum vapor stream to the first flash drum;
passing the second portion of the first flash drum liquid stream to the benzene toluene column at a first position;
passing the second flash drum liquid stream to the benzene-toluene column at a second position below the first position;
wherein the aromatics separation zone further comprises a benzene-toluene sidecut column, and further comprising:
passing an aromatics-rich stream from a sulfolane extraction process to the benzene-toluene sidecut column, the aromatics-rich stream comprising benzene and toluene;
separating the aromatics-rich stream from the extraction process in the benzene-toluene sidecut column into the benzene stream, the toluene stream, or both;
wherein the benzene stream comprises an overhead stream or a sidecut stream from the benzene-toluene sidecut column at a position above where the aromatics-rich stream from the extraction process enters the benzene-toluene sidecut column;
wherein the toluene stream comprises a bottom stream from the benzene-toluene sidecut column; and
wherein the xylenes stream comprises a bottom stream from the benzene-toluene column.

4. The process of claim 3 further comprising:
passing a sidecut stream from the benzene-toluene column to the benzene-toluene sidecut column at a position above a position where the aromatics-rich stream from the extraction process enters the benzene-toluene sidecut column; and
passing an overhead stream from the benzene-toluene sidecut column to the benzene-toluene column at a position above a position where the sidecut stream exits the benzene-toluene column;
wherein the benzene stream exits the benzene-toluene sidecut column at a position below the position where the sidecut stream from the benzene-toluene column enters the benzene-toluene sidecut column and above the position where the aromatic stream from the extraction process enters the benzene-toluene sidecut column.

5. The process of claim 3 further comprising:
dividing the hydrogen stream into a first portion and a second portion;
compressing the first portion of the hydrogen stream in a purge gas compressor forming a compressed first portion;
separating the compressed first portion of the hydrogen stream in a membrane separation unit into a permeate stream comprising hydrogen and a hydrogen-depleted offgas stream;
compressing the second portion of the hydrogen stream in a recycle gas compressor forming a compressed second portion;
combining the permeate stream and the compressed second portion forming a combined hydrogen stream; and
recycling the combined hydrogen stream to the combined feed stream.

6. The process of claim 1 further comprising:
precooling the reactor effluent stream with the combined feed stream before cooling the reactor effluent stream with the high-pressure liquid stream from the high-pressure product separator forming a precooled reactor effluent stream;
separating the precooled reactor effluent stream in a hot separator into a hot separator vapor stream and a hot separator liquid stream, wherein cooling the reactor effluent stream with the high-pressure liquid stream from the high-pressure product separator comprises cooling the hot separator vapor stream with the high-pressure liquid stream from the high-pressure product separator;
flashing the hot separator liquid stream in a second flash drum into a second flash drum vapor stream and a second flash drum liquid stream;
passing the second flash drum vapor stream to the first flash drum;
passing the second portion of the first flash drum liquid stream to the benzene-toluene column at a first position;
passing the second flash drum liquid stream to the benzene-toluene column at a second position below the first position;
wherein the aromatics separation zone further comprises a benzene-toluene sidecut column, and further comprising:
passing an aromatics-rich stream comprising benzene and toluene from a sulfolane extraction process to the benzene-toluene sidecut column;
separating the aromatics-rich stream in the benzene-toluene sidecut column into the benzene stream, the toluene column, or both;
wherein the benzene stream comprises an overhead stream or a sidecut stream from the benzene-toluene sidecut column at a position above where the aromatics-rich stream enters the benzene-toluene sidecut column;
wherein the toluene stream comprises a bottom stream from the benzene-toluene sidecut column; and
wherein the xylenes stream comprises a bottom stream from the benzene-toluene column.

7. The process of claim 6 further comprising:
dividing the hydrogen stream into a first portion and a second portion;
compressing the first portion of the hydrogen stream in a purge gas compressor forming a compressed first portion;
separating the compressed first portion of the hydrogen stream in a membrane separation unit into a permeate stream comprising hydrogen and a hydrogen-depleted offgas stream;
compressing the second portion of the hydrogen stream in a recycle gas compressor forming a compressed second portion;
combining the permeate stream and the compressed second portion of the hydrogen stream forming a combined hydrogen stream; and
recycling the combined hydrogen stream to the combined feed stream.

8. The process of claim 1 further comprising:
separating a benzene-toluene column overhead stream into an overhead offgas stream comprising hydrogen, light hydrocarbons having 1-5 carbon atoms, and part of the benzene, and an overhead liquid stream comprising hydrogen, light hydrocarbons having 1-5 carbon atoms, and a second part of the benzene;
compressing the overhead offgas stream forming a compressed overhead offgas stream;
passing the compressed overhead offgas stream to a stabilizer column; and
passing the overhead liquid stream to the stabilizer column.

9. The process of claim 1 further comprising:
compressing the hydrogen stream; and
recycling the compressed hydrogen stream to the combined feed stream.

10. The process of claim 1 further comprising:
preheating the combined feed stream in a heat exchanger with the reactor effluent stream, or a fired heater, or both before transalkylating the combined feed stream.

11. A process comprising:
transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen;
separating the reactor effluent stream in a hot separator into a hot separator vapor stream and a hot separator liquid stream;
cooling the hot separator vapor stream with a high-pressure liquid stream from a high-pressure product separator forming a cooled hot separator vapor stream and a heated high-pressure liquid stream;
separating the cooled hot separator vapor stream in the high pressure product separator forming a hydrogen stream and the high-pressure liquid stream from the high-pressure product separator;
flashing the hot separator liquid stream in a second flash drum into a second flash drum vapor stream and a second flash drum liquid stream;
flashing the heated high-pressure liquid stream and the second flash drum vapor stream in a first flash drum into a first flash drum vapor stream comprising the light hydrocarbons and a first flash drum liquid stream comprising the benzene, the xylenes, and the toluene;
passing the first flash drum liquid stream to an aromatics separation zone comprising a benzene-toluene column at a first position;
passing the second flash drum liquid stream to the benzene-toluene column at a second position below the first position; and
separating the first flash drum liquid stream and the second flash drum liquid stream into a benzene stream, or a toluene stream, or a xylenes stream, or combinations thereof in the aromatics separation zone.

12. The process of claim 11 further comprising:
precooling the reactor effluent stream with the combined feed stream before separating the reactor effluent stream in the hot separator forming a precooled reactor effluent stream;
wherein separating the reactor effluent stream in the hot separator comprises separating the precooled reactor effluent stream in the hot separator;
wherein the aromatics separation zone further comprises a benzene-toluene sidecut column, and further comprising:
passing an aromatics-rich stream comprising benzene and toluene from a sulfolane extraction process to the benzene-toluene sidecut column;
separating the aromatics-rich stream in the benzene-toluene sidecut column into the benzene stream, the toluene stream, or both;
wherein the benzene stream comprises an overhead stream or a sidecut stream from the benzene-toluene sidecut column at a position above where the aromatics-rich stream enters the benzene-toluene sidecut column;
wherein the toluene stream comprises a bottom stream from the benzene-toluene sidecut column; and
wherein the xylenes stream comprises a bottom stream from the benzene-toluene column.

13. The process of claim 12 further comprising:
passing a sidecut stream from the benzene-toluene column to the benzene-toluene sidecut column at a position above a position where the aromatics-rich stream enters the benzene-toluene sidecut column; and
passing an overhead stream from the benzene-toluene sidecut column to the benzene-toluene column at a position above a position where the sidecut stream exits the benzene-toluene column;
wherein the benzene stream exits the benzene-toluene sidecut column at a position below where the sidecut stream from the benzene-toluene column enters the benzene-toluene sidecut column.

14. The process of claim 11 further comprising:
dividing the hydrogen stream into a first portion and a second portion;
compressing the first portion of the hydrogen stream in a purge gas compressor forming a compressed first portion;
separating the compressed first portion of the hydrogen stream in a membrane separation unit into a permeate stream comprising hydrogen and a hydrogen depleted offgas stream;
compressing the second portion of the hydrogen stream in a recycle gas compressor forming a compressed second portion;
combining the permeate stream and the compressed second portion of the hydrogen stream forming a combined hydrogen stream; and
recycling the combined hydrogen stream to the combined feed stream.

15. The process of claim 11 further comprising:
compressing the hydrogen stream; and
recycling the compressed hydrogen stream to the combined feed stream.

16. The process of claim 11 further comprising:
preheating the combined feed stream in a heat exchanger with the reactor effluent stream, or a fired heater, or both before transalkylating the combined feed stream.

17. A process comprising:
transalkylating a combined feed stream comprising toluene, aromatic compounds having 9-11 carbon atoms, and hydrogen forming a reactor effluent stream comprising benzene, xylenes, toluene, unconverted aromatic compounds having 9-11 carbon atoms, light hydrocarbons having 1-5 carbon atoms, and hydrogen;
cooling the reactor effluent stream with a high pressure separator liquid stream from a high pressure product separator forming a cooled reactor effluent stream and a heated high pressure separator liquid stream;
separating the cooled reactor effluent stream in the high pressure product separator forming a hydrogen stream and the high pressure separator liquid stream;
heating the heated high pressure liquid stream with a stripper sidecut stream from a stripper column forming a second heated high pressure liquid stream and a cooled sidecut stream;
separating the second heated high pressure liquid stream in the stripper column into a stripper overhead stream comprising part of the benzene, non-aromatic compounds, and the light hydrocarbons, the stripper sidecut stream comprising benzene, xylenes, unconverted aromatic compounds, and toluene, and a stripper bottom stream comprising the benzene, the xylenes, the unconverted aromatic compounds, and the toluene, wherein the second heated high pressure stream enters the stripper column at a position above where the sidecut stream exits the stripper column;
combining the cooled stripper sidecut stream with the cooled reactor effluent stream; and
separating the stripper bottom stream into a benzene stream, or a toluene stream, or a xylenes stream, or combinations thereof in an aromatics separation zone.

18. The process of claim 17 further comprising:
heating the second heated liquid stream with the stripper bottom stream before separating the second heated high pressure liquid stream in the stripper column.

19. The process of claim 17 further comprising:
compressing the hydrogen stream; and
recycling the compressed hydrogen stream to the combined feed stream.

20. The process of claim 17 further comprising:
preheating the combined feed stream in a heat exchanger with the reactor effluent stream, or a fired heater, or both before transalkylating the combined feed stream.
